(19) United States
(12) Patent Application Publication
(10) Pub. No.: US 2012/0149731 A1
(43) Pub. Date: Jun. 14, 2012

(54) NEW MEDICAL USE

(75) Inventors: Kenneth Lyle Clark, Stevenage (GB); Edward Earl Philpot, Research Triangle Park, NC (US)

(73) Assignee: GLAXO GROUP LIMITED, Greenford, Middlesex (GB)

(21) Appl. No.: 13/378,299

(22) PCT Filed: Jun. 24, 2010
(86) PCT No.: PCT/EP2010/058979

§ 371 Date: Feb. 28, 2012

Related U.S. Application Data
(60) Provisional application No. 61/221,213, filed on Jun. 29, 2009.

(51) Int. Cl. A61K 31/4545; A61P 17/00

(52) U.S. Cl. 514/316

(57) ABSTRACT

The present invention relates to a method of treatment of urticaria comprising administration to a patient in need thereof an amount of the compound 3-(4-{[4-(4-{[3-(3,3-dimethyl-1-piperidinyl) propyl]oxy}phenyl)-1-piperidinyl]carbonyl}-1-naphthalenyl)propanoic acid,

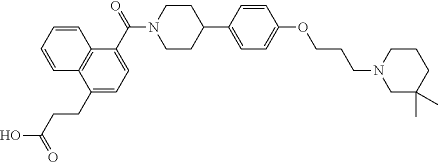

or a pharmaceutically acceptable salt thereof.

The present invention relates to the treatment of allergic skin diseases, in particular urticaria, chronic urticaria and chronic idiopathic urticaria with a compound which is 3-(4-{[4-(4-(4-{[3-(3,3-dimethyl-1-piperidinyl)propyl]oxy}phenyl)-1-piperidinyl]carbonyl}-1-naphthalenyl)propanoic acid or a pharmaceutically acceptable salt thereof.

Urticaria is one of the most common allergic dermatalogical conditions. The disease appears as a vascular reaction, characterised by red, raised, itchy circumscribed areas of dermal edema. The disease is classed as acute or chronic based on the persistence of the wheal and whether they do or do not spontaneously resolve. Deeper swellings of the skin (angiodema) can also occur which are painful rather than itchy. Wheals and angiodema may co-exist but either may occur alone. Chronic urticaria is a distressing condition with a very significant impact on patients' quality of life. The pathophysiology of urticaria is not well understood, however, an important factor in many patients in progression of the disease is the release of histamine from skin mast cells.

The physiological effects of histamine are classically mediated by four receptor subtypes, termed H₁, H₂, H₃ and H₄. The erythema, wheal formation anditching associated with urticaria are due to activation of H₁ receptors. Histamine H₂ receptors may also play a role in the wheal response produced by localized histamine since it has been demonstrated that H₂ antagonists attenuate the immediate vascular responses of intradermal (i.d.) injections of histamine. Combination treatment with a H₁ and H₂ antagonist is more effective in reducing the urticaria, itching and wheal and flare responses than treatment with either an H₁ or H₂ antagonist alone although the synergistic effect of combined H₁ and H₂ treatment for urticaria remains controversial since some investigators have not been able to demonstrate an improvement in chronic idiopathic urticaria with dual H₁ and H₂ treatment (see, for example, Commens C. A. & Greaves M. W., Brit. J. Dermatol., 1978, 99, 675-679; Cook L. J. & Shuster S. H., Acta Dermato-Venereologica (Stockh), 63, 265-267).

Histamine H₃ receptors are located presynaptically on postganglionic sympathetic noradrenergic nerves, including sympathetics innervating the blood vessels. Stimulation of H₃ receptors produces vasodilation by decreasing the release of noradrenaline from noradrenergic nerves terminals. McLeod et al. (Life Sciences, 2005, 76, 1784-94) studied in guinea pigs the vascular effects of endogenous mast cell histamine on H₃ receptors in the skin. These workers found that given together, a H₁ and H₃ antagonist attenuated skin responses produced by compound 48/80 to a greater extent than either a H₁ or H₃ antagonist alone in an experimental-induced urticaria model in guinea pigs. Therefore, molecules which are able to simultaneously block both histamine H₁ and H₃ receptors should prove to useful in reducing and preventing skin lesion formation in patients with urticaria and should prove to have superior efficacy to selective H₁ receptor antagonists commonly used to treat this disease. It is shown below that there is evidence for the presence of H3 receptors in the skin of patients suffering from chronic idiopathic urticaria, whereas a previous preclinical study using immunohistochemistry (Lippert et al., J. Invest. Dermatol., 2004, 123, 116-123) suggested that H3 receptors were not present in healthy human skin.

The present invention relates to the use of a compound which is 3-(4-{[4-(4-{[3-(3,3-dimethyl-1-piperidinyl) propyl]oxy}phenyl)-1-piperidinyl]carbonyl}-1-naphthalenyl)propanoic acid

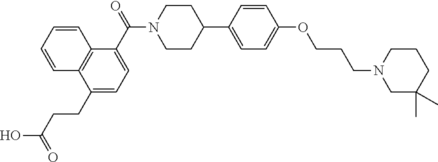

or a pharmaceutically acceptable salt thereof for the treatment of urticaria (e.g. chronic urticaria or chronic idiopathic urticaria).

4-{[4-(4-{[3-(3,3-dimethyl-1-piperidinyl)propyl]oxy}phenyl)-1-piperidinyl]carbonyl}-1-naphthalenyl) propanoic acid or a pharmaceutically acceptable salt thereof may show an improved profile over known dual H1/H3 receptor antagonists agonists in that it may possess one or more of the following properties:

(i) H3 receptor antagonist activity with a pKi of greater than about 7;
(ii) H1 receptor antagonist agonist activity with a pKi of greater than 7;
(iii) lower CNS penetration;
(iv) improved bioavailability; and
(v) lower clearance and/or longer half-life in blood.

This profile may be expected to be orally and/or topically effective, and/or capable of once daily administration and/or further may have an improved side effect profile compared with other existing therapies.

4-{[4-(4-{[3-(3,3-dimethyl-1-piperidinyl)propyl]oxy}phenyl)-1-piperidinyl]carbonyl}-1-naphthalenyl)propanoic acid or a pharmaceutically acceptable salt thereof may be in crystalline or amorphous form. Furthermore, this compound may exist in one or more polymorphic forms. Thus, the present invention includes within its scope the use of all polymorphic forms of 4-{[4-(4-{[3-(3,3-dimethyl-1-piperidinyl)propyl]oxy}phenyl)-1-piperidinyl]carbonyl}-1-naphthalenyl)propanoic acid or a pharmaceutically acceptable salt thereof. In general, the use of the most thermodynamically stable polymorphic form of 4-{[4-(4-{[3-(3,3-dimethyl-1-piperidinyl)propyl]oxy}phenyl)-1-piperidinyl]carbonyl}-1-naphthalenyl)propanoic acid or a pharmaceutically acceptable salt thereof is of particular interest.

Polymorphic forms of 4-{[4-(4-{[3-(3,3-dimethyl-1-piperidinyl)propyl]oxy}phenyl)-1-piperidinyl]carbonyl}-1-naphthalenyl)propanoic acid or a pharmaceutically acceptable salt thereof may be characterized and differentiated using a number of conventional analytical techniques, including but not limited to X-ray powder diffraction (XRPD) patterns, infrared (IR) spectra, Raman spectra, differential scanning calorimetry (DSC), thermogravimetric analysis (TGA) and solid state nuclear magnetic resonance (NMR).

It will be appreciated that many organic compounds can form solvates with the solvents in which they are reacted or from which they are precipitated or crystallized. For example, a solvate with water is known as a “hydrate”. Solvents with high boiling points and/or solvents with a high propensity to form hydrogen bonds such as water, xylene, N-methyl pyrrolidinone and methanol may be used to form solvates. Methods for identification of solvates include, but are not limited to, NMR and microanalysis. Thus, the use of solvates of 4-{[4-(4-{[3-(3,3-dimethyl-1-piperidinyl) propyl]oxy}phenyl)-1-piperidinyl]carbonyl}-1-naphthalenyl)propanoic acid or a pharmaceutically acceptable salt thereof are within the scope of the invention.

4-{[4-(4-{[3-(3,3-dimethyl-1-piperidinyl)propyl]oxy}phenyl)-1-piperidinyl]carbonyl}-1-naphthalenyl) propanoic acid may be in the form of and may be administered as a pharmaceutically acceptable salt. For a review on suitable salts see Berge et al., J. Pharm. Sci., 1977, 66, 1-19. Suitable pharmaceutically acceptable salts include acid and base addition salts.

Typically, a pharmaceutically acceptable salt may be readily prepared by using a desired acid or base as appropriate. The salt may precipitate from solution and be collected by filtration or may be recovered by evaporation of the solvent.

A pharmaceutically acceptable acid addition salt can be formed by reaction of 4-{[4-(4-{[3-(3,3-dimethyl-1-piperidinyl) propyl]oxy}phenyl)-1-piperidinyl]carbonyl}-1-naphthalenyl)propanoic acid with a suitable inorganic or organic acid (such as hydrobromic, hydrochloric, formic, sulfuric, nitric, phosphoric, succinic, maleic, acetic, fumaric, citric, tartaric, benzoic, p-toluenesulfonic, methanesulfonic or naphthalenesulfonic acid), optionally in a suitable solvent such as an organic solvent, to give the salt which is usually isolated for example by crystallisation and filtration. Thus, a pharmaceutically acceptable acid addition salt of 4-{[4-(4-{[3-(3,3-dimethyl-1-piperidinyl) propyl]oxy}phenyl)-1-piperidinyl]carbonyl}-1-naphthalenyl)propanoic acid can be for example a hydrobromide, hydrochloride, formate, sulfate, nitrate, phosphate, succinate, maleate, acetate, fumarate, citrate, tartrate, benzoate, p-toluenesulfonate, methanesulfonate or naphthalenesulfonate salt.

The hydrochloride salt of 4-{[4-(4-{[3-(3,3-dimethyl-1-piperidinyl)propyl]oxy}phenyl)-1-piperidinyl]carbonyl}-1-naphthalenyl) propanoic acid is of particular interest.

A pharmaceutically acceptable base addition salt can be formed by reaction of a 4-{[4-(4-{[3-(3,3-dimethyl-1-piperidinyl) propyl]oxy}phenyl)-1-piperidinyl]carbonyl}-1-naphthalenyl)propanoic acid with a suitable inorganic (e.g. ammonia) or organic base (e.g. triethylamine, ethanolamine, triethanolamine, choline, arginine, lysine or histidine), optionally in a suitable solvent such as an organic solvent, to give the base addition salt which is usually isolated for example by crystallisation and filtration.

Other suitable pharmaceutically acceptable salts include pharmaceutically acceptable metal salts, for example pharmaceutically acceptable alkali-metal or alkaline-earth-metal salts such as sodium, potassium, calcium or magnesium salts; in particular pharmaceutically acceptable metal salts of the carboxylic acid moieties that is present in 4-{[4-(4-{[3-(3,3-dimethyl-1-piperidinyl)propyl]oxy}phenyl)-1-piperidinyl]carbonyl}-1-naphthalenyl)propanoic acid.

Included within the scope of the invention is the use of all solvates e.g. hydrates and polymorphs of 4-{[4-(4-{[3-(3,3-dimethyl-1-piperidinyl)propyl]oxy}phenyl)-1 -piperidinyl]carbonyl}-1-naphthalenyl) propanoic acid or the pharmaceutically acceptable salts thereof in the treatment of urticaria (e.g. chronic urticaria or chronic idiopathic urticaria).

4-{[4-(4-{[3-(3,3-dimethyl-1-piperidinyl)propyl]oxy}phenyl)-1-piperidinyl]carbonyl}-1-naphthalenyl) propanoic acid or a pharmaceutically acceptable salt thereof may be prepared according to the general methods and experimental section provided in International Patent Application Publication Number WO2007/071691 (U.S. application Ser. No. 12/158185) (see in particular Example 3) which is incorporated herein by reference.

4-{[4-(4-{[3-(3,3-dimethyl-1-piperidinyl)propyl]oxy}phenyl)-1-piperidinyl]carbonyl}-1 -naphthalenyl) propanoic acid or a pharmaceutically acceptable salt thereof is expected to have beneficial anti-inflammatory and/or anti-allergic effects and therefore may be of use in the treatment of urticaria (e.g. chronic urticaria or chronic idiopathic urticaria).

It will be appreciated by those skilled in the art that references herein to treatment or therapy may extend to prophylaxis as well as the treatment of established conditions.

There is thus provided the use of 4-{[4-(4-{[3-(3,3-dimethyl-1-piperidinyl)propyl]oxy}phenyl)-1-piperidinyl]carbonyl}-1-naphthalenyl)propanoic acid or a pharmaceutically acceptable salt thereof in the treatment of urticaria (e.g. chronic urticaria or chronic idiopathic urticaria).

There is also provided the use of 4-{[4-(4-{[3-(3,3-dimethyl-1-piperidinyl)propyl]oxy}phenyl)-1-piperidinyl]carbonyl}-1-naphthalenyl)propanoic acid or a pharmaceutically acceptable salt thereof for the manufacture of a medicament for the treatment of urticaria (e.g. chronic urticaria or chronic idiopathic urticaria).

In a further aspect of the invention, there is provided a method for the treatment (or prophylaxis) of urticaria (e.g. chronic urticaria or chronic idiopathic urticaria), in a patient in need thereof, which method comprises administering an effective amount of 4-{[4-(4-{[3-(3,3-dimethyl-1-piperidinyl) propyl]oxy}phenyl)-1-piperidinyl]carbonyl}-1-naphthalenyl)propanoic acid or a pharmaceutically acceptable salt thereof.

### ROUTES OF ADMINISTRATION

When used in therapy, 4-{[4-(4-{[3-(3,3-dimethyl-1-piperidinyl)propyl]oxy}phenyl)-1-piperidinyl]carbonyl}-1-naphthalenyl)propanoic acid or a pharmaceutically acceptable salt thereof is usually formulated in a suitable pharmaceutical composition. Such pharmaceutical compositions can be prepared using standard procedures.

Thus, the present invention further provides a composition which comprises a compound which is 4-{[4-(4-{[3-(3,3-dimethyl-1-piperidinyl) propyl]oxy}phenyl)-1-piperidinyl]carbonyl}-1-naphthalenyl) propanoic acid or a pharmaceutically acceptable salt thereof optionally with one or more pharmaceutically acceptable carriers and/or excipients for use in the treatment of urticaria, e.g. chronic urticaria or chronic idiopathic urticaria.

A composition comprising 4-{[4-(4-{[3-(3,3-dimethyl-1-piperidinyl)propyl]oxy}phenyl)-1-piperidinyl]carbonyl}-1-naphthalenyl)propanoic acid or a pharmaceutically acceptable salt thereof, which may be prepared by admixture, suitably at ambient temperature and atmospheric pressure, may be adapted for oral, parenteral, rectal or intranasal administration and, as such, may be in the form of tablets, capsules, liquid preparations e.g. oral liquid preparations, powders, granules, lozenges, reconstitutable powders, injectable or infusible solutions or suspensions or suppositories. Suitable compositions may be prepared according to methods well known in the art for each particular type of composition.

Compositions suitable for oral administration are of particular interest. Another composition of interest is a composition suitable for topical administration. Thus, in one aspect of the invention, 4-{[4-(4-{[3-(3,3-dimethyl-1-piperidinyl) propyl]oxy}phenyl)-1-piperidinyl]carbonyl}-1-naphthalenyl) propanoic acid or a pharmaceutically acceptable salt thereof is administered orally or topically.

The compositions may contain from about 0.1% to 99% by weight, such as from about 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be about 0.05 to 1000 mg, more suitably about 1.0 to 200 mg, for example 20 to 100 mg, and such unit doses may be administered more than once a day, for example two or three times a day. Such therapy may extend for a number of weeks or months. In one embodiment, compounds and pharmaceutical compositions according to the invention are suitable for oral administration and/or are capable of once daily administration, for example at a dose in the range of 20 to 200 mg (e.g. about 20 to 100 mg, such as about 10 to 50 mg).

### Orally Administered Compositions

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders suitable for incorporating into tablets or capsules may be prepared by reducing the compound to a suitable fine size (e.g. by micronisation) and mixing with a similarly prepared pharmaceutical carrier such as an edible carbohydrate, as, for example, starch or mannitol. Flavoring, preservative, dispersing and coloring agent can also be present.

Capsules may be made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, glidants, lubricants, sweetening agents, flavours, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressedinto tablets. The compound or salt can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylatedisostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit compositions for oral administration can be microencapsulated. The composition can also be prepared to prolong or sustain the release as for example by coating or embedding particulate material in polymers, wax or the like.

For stability purposes, the composition of the present invention may be protected from microbial contamination and growth by inclusion of a preservative. Examples of pharmaceutically acceptable anti-microbial agents or preservatives may include quaternary ammonium compounds (e.g. benzalkonium chloride, benzethonium chloride, cetrimide and cetylpyridinium chloride), mercurial agents (e.g. phenylmercuric nitrate, phenylmercuric acetate and thimerosal), alcoholic agents (e.g. chlorobutanol, phenylethyl alcohol and benzyl alcohol), antibacterial esters (e.g. esters of para-hydroxybenzoic acid), chelating agents such as disodium edetate (EDTA) and other anti-microbial agents such as chlorhexidine, chlorocresol, sorbic acid and its salts and polymyxin.

Compositions which contain a suspended medicament may include a pharmaceutically acceptable wetting agent which functions to wet the particles of medicament to facilitate dispersion thereof in the aqueous phase of the composition. Typically, the amount of wetting agent used will not cause foaming of the dispersion during mixing. Examples of wetting agents include fatty alcohols, esters and ethers, such as polyoxyethylene (20) sorbitan monooleate (Polysorbate 80).

A tonicity adjusting agent may be included to achieve isotonicity with body fluids resulting in reduced levels of irritancy. Examples of tonicity adjusting agents include sodium chloride, dextrose and calcium chloride.

### Externally Administered Topical Compositions

The composition can for example be adapted for topical administration, such as external topical administration (e.g. topical administration to the skin). External topical administration can for example be to those parts of the skin affected by or susceptible to the disease or condition e.g. urticaria.

An external-topical composition, e.g. skin topical pharmaceutical composition, can for example be an ointment, a cream (usually an oil-in-water or water-in-oil pharmaceutical composition, which is usually an emulsion), an aqueous gel, or a microemulsion. The composition can alternatively be a DMSO-containing solution such as a DMSO/acetone solution or DMSO/water solution (DMSO=dimethyl sulfoxide); a DMSO-containing solution can be used for experimental animal tests, but is not usually desirable for use in humans.

In the external-topical pharmaceutical composition, e.g. an ointment or an oil-in-water or water-in-oil composition, 4-{[4-(4-{[3-(3,3-dimethyl-1-piperidinyl)propyl]oxy}phenyl)-1-piperidinyl]carbonyl}-1-naphthalenyl) propanoic acid or a pharmaceutically acceptable salt thereof can be present in 0.1% to 10%, such as 0.2% to 10%, or 0.2% to 5%, or 0.5% to 5%, in particular 1% to 10% (e.g. about 2%, about 4% or about 6%), or 1% to 5% (e.g. 1.5% to 5% or 1.5% to 5%, such as about 2% or about 4%), or 0.5% to 3% (e.g. 0.5% or about 2%), or 1% to 3% (e.g. about 2%), by weight of the composition (w/w).

In the external-topical pharmaceutical composition of the invention, 4-{[4-(4-{[3-(3,3-dimethyl-1-piperidinyl) propyl]oxy}phenyl)-1-piperidinyl]carbonyl}-1-naphthalenyl)propanoic can in particular be the hydrochloride salt.

4-{[4-(4-{[3-(3,3-Dimethyl-1-piperidinyl)propyl]oxy}phenyl)-1-piperidinyl]carbonyl}-1-naphthalenyl) propanoic acid or a pharmaceutically acceptable salt thereof can optionally be in a particle-size-reduced form, for example obtained or obtainable by micronisation. This can be, for example, for use in a pharmaceutical composition adapted for topical, such as external topical (e.g. skin topical) administration.

Aqueous solubility: A preliminary screen, which can aim to estimate roughly the aqueous solubility of 4-{[4-(4-{[3-(3,3-dimethyl-1-piperidinyl)propyl]oxy}phenyl)-1-piperidinyl]carbonyl}-1-naphthalenyl) propanoic acid or a pharmaceutically acceptable salt thereof can include (as an approximate summary): (i) creating an approximately 10mM solution of the compound in DMSO, (ii) diluting a portion of this DMSO solution by mixing about 19 parts by volume of pH 7.4 aqueous phosphate buffered saline (PBS) buffer with 1 part by volume of the approximate 10mM DMSO solution, (iii) “filtering” the mixture with the aid of centrifugation, and then (iv) measuring the concentration of the dissolved compound in the “filtrate”. Although some DMSO (about 5% by volume) is usually present in this solubility screen “filtrate”, the results can be a very approximate estimate of aqueous solubility, e.g. at room temperature.

Lipophilicity: The clogP (calculated log of the octanol/water partition coeficient (P)) of 4-{[4-(4-{[3-(3,3-dimethyl-1-piperidinyl) propyl]oxy}phenyl)-1-piperidinyl]carbonyl}-1-naphthalenyl) propanoic acid or a pharmaceutically acceptable salt thereof can estimate the lipophilicity of the compound or salt.

Solubilising and/or skin-penetration-enhancing agents: An external-topical pharmaceutical composition, e.g. an ointment or an oil-in-water cream or water-in-oil cream, can for example include an agent which acts as a skin-penetration enhancer for and/or a solubiliser of 4-{[4-(4-{[3-(3,3-dimethyl-1-piperidinyl)propyl]oxy}phenyl)-1-piperidinyl]carbonyl}-1-naphthalenyl) propanoic acid or a pharmaceutically acceptable salt thereof. The skin-penetration-enhancing—and/or solubilising—agent can for example be propylene glycol, diethylene glycol monoethyl ether (e.g. TRANSCUTOL™) and/or caprylocaproyl macrogolglycerides (e.g. LABRASOL™), in particular propylene glycol. The solubiliser and/or skin-penetration enhancer suitably does not comprise DMSO. The solubiliser and/or skin-penetration enhancer is in particular both a solubiliser and skin-penetration enhancer, and/or can be present in 0.5% to 50%, in particular 5% to 50%, for example 7% to 30%, such as 7% to 25%, e.g. about 10% to about 20% (e.g. about 10% or about 20%), by weight of the composition (w/w).

The skin-penetration enhancer is for delivery of 4-{[4-(4-{[3-(3,3-dimethyl-1-piperidinyl) propyl]oxy}phenyl)-1-piperidinyl]carbonyl}-1-naphthalenyl)propanoic acid or a pharmaceutically acceptable salt thereof through the skin. Solubilization of the compound or salt also helps. The solubilising and/or skin-penetration-enhancing agents shouldideally (a) be safe and/or tolerable, (b) have as low a potential for skin irritancy as possible consistent with being an effective skin penetration enhancer, and (c) be compatibile with the active ingredient. Note that the agent optionally functions both as a solubilising agent and a skin-penetration-enhancing agent.

Surfactants: An external-topical pharmaceutical composition, e.g. an ointment or an oil-in-water cream or water-in-oil cream, can include a surfactant (e.g. as an emulsifier), for example for achieving emulsification of compositions having two or more phases. The total surfactant content can for example be 0.3% to 20%, e.g. 0.5% to 15% or 0.5% to 12% or 0.5% to 10% or 1% to 12% or 3% to 10%, by weight of the composition (w/w). The surfactant can for example comprise one or more of the following: a polyoxyl C₁₂₋₂₂alkyl ether (e.g. a polyoxyl C₁₄₋₂₀alkyl ether such as polyoxyl cetyl ether or polyoxyl stearyl ether) (e.g. present at 0.5% to 10% w/w, e.g. 2.5% to 10% w/w such as about 5% to about 8% w/w), glycerol monostearate (e.g. Arlacel 165™) (e.g. present at 0.5% to 10% w/w, e.g. about 2% w/w), sorbitan monostearate (e.g. Span 60™) (e.g. present at 0.05% to 10% w/w, e.g. about 1% w/w), cetyl alcohol and/or stearyl alcohol (e.g. wherein the total of any cetyl alcohol and any stearyl alcohol present is 0.1% to 15% w/w, e.g. 1% to 10% w/w such as about 2% to about 5% w/w), and sodium dodecyl sulphate (SDS) (e.g. present at 0.3% to 2% w/w such as about 1% w/w). Polyoxyl stearyl ether (steareth) can e.g. be polyoxyl 2 stearyl ether (steareth 2) or polyoxyl 21 stearyl ether (steareth 21).

DMSO-containing solutions: One possible external-topical composition is a solution of 4-{[4-(4-{[3-(3,3-dimethyl-1-piperidinyl) propyl]oxy}phenyl)-1-piperidinyl]carbonyl}-1-naphthalenyl) propanoic acid or a pharmaceutically acceptable salt thereof present at about 0.5% to about 2.5% w/w in a DMSO-containing solvent such as in DMSO/acetone or in DMSO/water; for example a solution of the compound or salt present at about 0.5% to about 2.5% w/w in DMSO/acetone (1:1). DMSO-containing solutions, often being capable of high skin penetration, are often good experimental pre-clinical formulations for use in animals e.g. pigs, but their likely skin irritancy generally make them less suitable for use in humans such as patients, e.g. atopic dermatitis patients.

### Ointments (and Oil Phase in Ointments and Creams):

An external-topical composition can for example be an ointment or an oil-in-water cream or water-in-oil cream. The ointment or cream typically contains an oil phase (oily ointment base). The oil phase (oily ointment base) typically comprises an oil and/or a fat, for example of a consistency suitable for skin-spreadability.

The oil phase (oily ointment base) can for example comprise or be an oil, wherein the oil comprises or is white soft paraffin (white petrolatum) and/or a silicone oil and/or a mineral oil (such as liquid paraffin). Mineral oil can also be used as a solubiliser and/or emollient.

In particular, the oil phase (oily ointment base) comprises or is an oil, wherein the oil comprises or is white soft paraffin (white petrolatum) and/or a silicone oil.

The white soft paraffin (white petrolatum), e.g. in an ointment or cream, can be of various grades, for example (for Penreco supplier) Penreco Regent White™ grade, Penreco Snow White™ grade, or Penreco Ultima White™ grade; in particular high melting point white petrolatum (high melting point white soft paraffin) (e.g. of Penreco Ultima White ™ grade). The white petrolatum can be present at 25% to 99.9% w/w or 45% to 99.5% w/w or 50% to 99.5% w/w or 45% to 99% w/w or 50% to 99% w/w or 45% to 85% w/w or 45% to 75% w/w.

The silicone oil, e.g. in an ointment or cream can for example be present at: 5% to 60% w/w such as 5% to 50% w/w, in particular 10% to 50% w/w such as 15% to 40% w/w, suitably 20% to 35% w/w such as about 25% w/w.

The silicone oil can be solid or liquid. The silicone oil, e.g. in an ointment or cream, can for example comprise or be: decamethyl-cyclopentasiloxane (e.g. ST-Cyclomethicone 5-NF™, available from Dow Corning), stearoxytrimethylsilane [Me(CH₂)₁₇O—SiMe₃], polydimethylsiloxane (dimethicone), hexamethyldisiloxane (e.g. about 0.65 cSt viscosity at 25° C.), octamethyltrisiloxane (e.g. about 1.0 cSt viscosity at 25° C.), decamethyltetrasiloxane, dodecamethylpentasiloxane, or hydroxy-terminated polydimethylsiloxane (e.g. ST-Dimethiconol 40™, Dow Corning), or mixtures of any of the foregoing. The silicone oil, e.g. in an ointment or cream, can in particular comprise or be: decamethyl-cyclopentasiloxane, stearoxytrimethylsilane [Me(CH₂)₁₇O—SiMe₃], or polydimethylsiloxane (dimethicone), or mixtures of any of the foregoing. Preferably, the silicone oil, e.g. in an ointment or cream, can comprise or be decamethyl-cyclopentasiloxane.

The decamethyl-cyclopentasiloxane can be ST-Cyclomethicone 5-NF ™, available from Dow Corning, and which is described by Dow Corning as being a polydimethyl-cyclosiloxane having a decamethyl-cyclopentasiloxane content of >95% and having a octamethyl-cyclotetrasiloxane content of <1.0%. The decamethyl-cyclopentasiloxane can for example be present at 5% to 60% w/w such as 5% to 50% w/w, in particular 10% to 50% w/w such as 15% to 40% w/w, suitably 20% to 35% w/w such as about 25% w/w.

Stearoxytrimethylsilane [Me(CH₂)₁₇O—SiMe₃] can for example be present as a mixture of stearoxytrimethylsilane and stearyl alcohol for example Silky Wax 10™ which is available from Dow Corning. Stearoxytrimethylsilane (and/or stearoxytrimethylsilane and stearyl alcohol mixture), e.g. in an ointment or cream, can for example be present at 1% to 30% w/w or 2% to 20% w/w or 5% to 20% w/w such as about 10% w/w.

Polydimethylsiloxane (dimethicone), whose structure is given in the Merck Index 12th edition 1996 as Me₃Si—O—[—Si(CH₃)₂—O]ₙ—SiMe₃, can for example have a viscosity at 25° C. of from about 20 to about 12500 cSt (centistokes), such as a viscosity at 25° C. of from about 20 to about 350 cSt or from about 20 to about 100 cSt. For example, polydimethylsiloxane (dimethicone) can have a viscosity at 25° C. of: 20 cSt (±10%) (“dimethicone 20”), 100 cSt (±5%), 350 cSt (±5%) (“dimethicone 350”), 1000 cSt (±5%), or 12500 cSt (±5%); grades of polydimethylsiloxane having these five different viscosities are available from Dow Corning as Q7-9120™ Silicone Fluid. Polydimethylsiloxane (dimethicone), e.g. in an ointment, can e.g. be present at 0.1% to 15% w/w such as 0.5% to 10% w/w e.g. 0.5% to 5% w/w.

Microcrystalline wax or beeswax or beeswax substitute can alternatively or additionally be used as an oil/fat in the oil phase.

Alternatively or additionally, one or more fats, e.g. straight or branched chain mono- or di-alkyl esters such as isopropyl myristate, isopropyl palmitate, diisopropyl adipate, isocetyl stearate, isostearyl isostearate, decyl oleate, butyl stearate, 2-ethylhexyl palmitate, propylene glycol diester of coconut fatty acids, or a mixed ester of 2-ethyl hexanoic acid with a blend of cetyl or stearyl alcohols (e.g.

known as Crodamol CAP) may be used in the oil phase (some of these are also solubilisers and/or surfactants). These may be used singly or in combination depending on the properties required.

The oil phase (oily ointment base) can for example be present at 25% to 99.9% w/w or 25% to 99.5% w/w or 25% to 85% w/w (in particular 45% to 99.5% w/w or 45% to 99% w/w, or 50% to 99.5% w/w or 50% to 99% w/w or 50% to 80% w/w, or 70% to 99.5% w/w or 80% to 99.5% w/w) in an ointment (e.g. as an emulsion, or e.g. as a homogeneous single phase (which does not exclude the compound or salt being at least partly in suspension)).

The oil phase (oily ointment base) can for example be present at 25% to 85% w/w (e.g. 35% to 70% w/w) in an water-in-oil cream (e.g. emulsion), or at 8% to 55% w/w (e.g. 10% to 45% w/w) in an oil-in-water cream (e.g. emulsion).

Ointment compositions having two phases can optionally be prepared using an emulsification process whereby the hydrophilic phase (e.g. propylene-glycol-containing phase) and oil phase are first prepared in separate vessels. The hydrophilic phase can optionally contain a penetration enhancer such as propylene glycol, and optionally some or all of the compound of formula (I) or salt thereof. The oil phase can optionally contain a surfactant. Temperatures of both phases are maintained at elevated temperatures, such as about 45-90° C. or about 45-80° C. or about 55-90° C. or about 55-80° C. (e.g. about 60-65° C.), or from above 70 to 90° C., the oil phase temperature being sufficiently high (e.g. from above 70 to 90° C.) to melt the oil phase. While hot, one phase is added to another while mixing, e.g. using a high shear mixer, to effect emulsification, optionally keeping the temperature above 70° C. such as from above 70 to 90° C. The resulting ointment emulsion is allowed to cool, e.g. to about 15-35° C. such as to about 17-30° C., in particular while the agitation continues e.g. at lower speeds. The ointment emulsion can then optionally be dispensed from the manufacturing vessel and filled into primary packaging, for example tubes or sachets.

Optionally, an ointment can comprise a polyethylene glycol base, e.g. present at 25 to 98% w/w such as 50 to 95% w/w, instead of or as well as an oily ointment base.

Creams: An external-topical composition can be a cream, e.g. a water-in-oil cream or an oil-in-water cream.

Water-in-oil creams: These usually have an increased aqueous content compared to ointments. In particular, the water-in-oil cream can be a water-in-oil cream emulsion. That is, in particular, in the water-in-oil cream, an oil phase and an aqueous phase can have been emulsified to form a water-in-oil cream emulsion.

Oil-in-water creams: These usually have an increased aqueous content compared to ointments and water-in-oil creams. In particular, the oil-in-water cream can be an oil-in-water cream emulsion. That is, in particular, in the oil-in-water cream, an oil phase and an aqueous phase can have been emulsified to form an oil-in-water cream emulsion.

Oil-in-water creams can for example be high-occlusion creams, wherein, after topical administration to the skin, moisture loss from the skin and/or from the cream is reduced or limited by means of sufficiently high coverage of the skin and/or by providing a sufficient barrier at the site of application.

An oil-in-water cream can in particular contain one or more emollients (hydrating agents), such as silicones (e.g. dimethicone, e.g. dimethicone 360 or dimethicone 20), a high-viscosity wax such as microcrystalline wax, and/or mineral oil.

In an oil-in-water cream, suitably there is a sufficiently high water content, for example wherein the water is present in 15% to 60% w/w, 20% to 50% w/w, or 25% to 40% w/w.

Cream emulsions, e.g. water-in-oil or oil-in-water cream emulsions, can generally be prepared by a process in which an aqueous phase is prepared, e.g. prepared before emulsification. The aqueous phase usually contains water and a solubiliser and/or skin-penetration enhancer such as propylene glycol, and optionally contains some or all of -{[4-(4-{[3-(3,3-dimethyl-1-piperidinyl)propyl]oxy}phenyl)-1-piperidinyl]carbonyl}-1-naphthalenyl)propanoic acid or a pharmaceutically acceptable salt thereof, and/or optionally contains surfactant. The oil phase, e.g. containing white petrolatum and/or mineral oil, and/or optionally containing surfactant, can be prepared in a separate vessel. Temperatures of both phases are suitably maintained at (or heated to) elevated temperatures, such as about 45-90° C. or about 45-80° C. or about 45-75° C., for example about 55-90° C. or about 55-80° C. or about 55-75° C. (in particular at about 60-65° C.), or e.g. from above 70 to 90° C., the oil phase temperature being sufficiently high (e.g. about 45-90° C. or about 55-90° C. or from above 70 to 90° C.) to melt the oil phase. While hot, one phase is suitably added to another while mixing, e.g. using a high shear mixer, to effect emulsification, for example keeping the temperature 45° C. or above, or 55° C. or above such as above 70° C. e.g. from above 70 to 90° C. The resulting emulsion is typically allowed to cool, e.g. to about 15-35° C. such as to about 17-30° C. (e.g. to about 17-22° C.) or to about 18-30° C., for example while the agitation continues e.g. at lower speeds. The cream emulsion can then optionally be dispensed from the manufacturing vessel and filled into primary packaging, for example tubes or sachets.

Typically, a pharmaceutical composition of the invention suitable for external topical administration can be administered once daily, twice daily or more than twice daily, to external body part(s), e.g. on the skin such as at a site of diseased skin, e.g. skin suffering from atopic dermatitis.

### Combination Products

The compounds and pharmaceutical compositions herein may also be used in combination with or include one or more other therapeutic agents, for example anti-inflammatory agents such as steroids (oral and/or topical) e.g. corticosteroids; non-steroidal anti-inflammatory drugs (NSAIDs) (e.g. diclofenac, ibuprofen, aspirin); oral immunosuppresive drugs (e.g. methotrexate, cyclosporine); anti-IgE inhibitors (e.g. omalizumab); leukotriene antagonists (e.g. montelukast) andinhibitors of leukotriene synthesis; inhibitors of mast cell activation (e.g. nedocromil sodium, sodium cromoglycate) or inhibitors of prostagland in synthesis or prostagland in antagonists.

It will be clear to a person skilled in the art that, where appropriate, the other therapeutic agent(s) may be used in the form of salts, (e.g. as alkali metal or amine salts or as acid addition salts), or prodrugs, or as esters (e.g. lower alkyl esters), or as solvates (e.g. hydrates) to optimise the activity and/or stability and/or physical characteristics (e.g. solubility) of the therapeutic agent. It will be clear also that where appropriate, the therapeutic agents may be used in optically pure form.

Investigations into Presence of H3 Receptors in Skin of Patients Suffering from Urticaria

### Background and Results:

Although preclinical studies (McLeod et al. (Life Sciences, 2005, 76, 1784-94)) suggest that H3 receptors are expressed in guinea pig skin, it has not been clear whether H3 receptors are expressed in healthy or diseased human skin. Very few studies have addressed this question. One previous study using immunohistochemistry in healthy human skin (Lippert et al., J. Invest. Dermatol., 2004, 123, 116-123) failed to find evidence for the presence of H3 receptors. However, no previously published studies have evaluated whether the H3 receptor is present in skin samples from urticaria patients. Therefore, using immunohistochemistry, we sought to clarify whether the H3 receptor is expressed in healthy human skin and in lesional and non-lesional skin samples from urticaria patients. Our novel findings indicate that in contrast to Lippert et al., we find positive evidence for the expression of the histamine H3 receptor in both human healthy abdominal skin and in lesional and non-lesional skin samples from urticaria patients. Similar to human normal abdominal skin samples, the data from the urticaria samples support the presence of the H3 receptor in the epidermis (keratinocytes). There also appears to be H3 receptor expression associated with blood vessels (endothelial), nerves and with inflammatory cells suspected to be mast cells. Some weak smooth muscle staining was also observed. The expression pattern appears similar in both lesional and non-lesional skin although there appears to be greater vascular staining in the lesions. The expression pattern of the H3 receptor in non-lesional and lesional skin from urticaria patients was similar to that of the H1 receptor. Overall, these data are consistent with a possible role for the H3 receptor in mediating the itch, redness, inflammation and wheal formation that occur following the release of histamine and are consistent with the hypothesis that dual blockade of H1 and H3 receptors with a single molecule in poorly treated skin disorders such as urticaria will offer greater efficacy than either a selective H1 or H3 antagonist given in isolation.

### Methodology:

All skin samples were collected with full informed ethical consent. Samples were fixed in buffered formalin underwent routine histological processing. The paraffin wax embedded samples were sectioned at 4 microns onto glass slides. Standard immunohistochemistry (IHC) was then carried out using Bond Leica automated staining. Epitope retrieval using ERI buffer was employed followed by incubation of the sections in either commercially sourced polyclonal H3 receptor or H1 receptor primary antibody. Negative controls were run on adjacent sections using a rabbit IgG isotype reagent at similar dilution to the antibody titre. Coverslipped slides were scanned using the Hamamatsu NanoZoomer and each digital image was examined for H3 receptor and H1 receptor positive staining in each of the prepared skin sections. Examination of the sections showed good morphology and each sample had adequate areas of epidermis and sub cutis to allow interpretation.

1-12. (canceled)

13. A method for the treatment of urticaria which comprises administering to a patient in need thereof an effective amount of a compound which is 3-(4-{[4-(4-{[3-(3,3-dimethyl-1-piperidinyl)propyl]oxy}phenyl)-1-piperidinyl ]carbonyl}-1-1-naphthalenyl)propanoic acid, 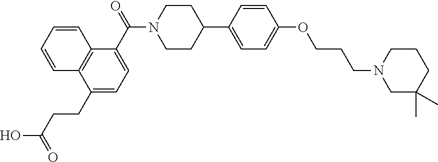
or a pharmaceutically acceptable salt thereof.

14. The method according to claim 13 wherein the compound is in the form of a hydrochloride salt.

15. The method according to claim 13, wherein the urticaria is of chronic urticaria.

16. The method according claim 15, wherein the chronic urticaria is of chronic idiopathic urticaria.

17. A method according to claims 13 wherein the compound is administered orally.

18. A method according to claim 13 wherein the compound is administered topically.

19. The method according to claim 14, wherein the urticaria is chronic urticaria.

20. The method according claim 19, wherein the chronic urticaria is chronic idiopathic urticaria.

21. A method according to claim 14 wherein the compound is administered orally.

22. A method according to claim 14 wherein the compound is administered topically.
